(19) United States
(12) Patent Application Publication
(10) Pub. No.: US 2026/0137875 A1
(43) Pub. Date: May 21, 2026

(54) Needle Guard for a Medical Syringe

(71) Applicants: Thomas J. Shaw, Frisco, TX (US); Retractable Technologies, Inc., Little Elm, TX (US)

(72) Inventors: Thomas J. Shaw, Frisco, TX (US); Jordan Duesman, Pilot Point, TX (US)

(21) Appl. No.: 19/387,051

(22) Filed: Nov. 12, 2025

Related U.S. Application Data
(60) Provisional application No. 63/722,167, filed on Nov. 19, 2024.

(51) Int. Cl. A61M 5/32

(52) U.S. Cl. CPC A61M 5/3202

(57) ABSTRACT

Systems and methods are disclosed that include providing a needle guard for a syringe. The needle guard includes a main body forming an open top receiving enclosure that allows a syringe to be inserted into the main body through the open top. The main body includes an opening formed in a front wall through which the syringe may extend. Components of the main body engage the syringe and the restrict movement of the syringe when the syringe is disposed within the needle guard. The needle guard includes a lid hingedly coupled to the main body. The main body and the lid include complementary latch components that secure the lid to the main body in a closed position. The needle guard allows a user to apply a longitudinal extraction force to the syringe to remove the syringe from the needle guard through the opening formed in the front wall of the needle guard.

### CROSS-REFERENCE TO RELATED APPLICATIONS

This application claims priority under 35 U.S.C. § 119(e) to U.S. Provisional Ser. No. 63/722,167 filed on Nov. 19, 2024 by Thomas J. Shaw et al. and entitled “Needle Guard For a Medical Syringe,” the disclosure of which is hereby incorporated by reference in its entirety for all purposes.

### BACKGROUND OF THE INVENTION

Medical syringes having a hypodermic needle are commonly used for the administration of injections and/or infusions of drugs or medicines. Medical syringes are commonly shipped and transported to medical facilities and/or end users with a removable protective needle cap that covers the hypodermic needle to protect the needle from contamination and damage. Traditional needle caps simply slide on over the needle and are simply held in place by friction for easy removal prior to administration of an injection. However, such needle caps are prone to loosening and/or falling off, which can result in damage or contamination to the needle, thereby rendering the associated syringe unusable. Accordingly, the medical industry continues to demand advances in needle guard technology that more securely and effectively protect the needle from such damage and contamination, while maintaining and/or enhancing useability of the syringes.

### SUMMARY

In some embodiments, a needle guard is disclosed as comprising: a main body forming an open top receiving enclosure configured to at least partially receive a syringe, wherein the main body includes an open front portion including an opening formed in a front wall, a barrel retaining guide disposed in proximity to the front wall and including an arcuate aperture configured to engage a portion of a barrel of the syringe, and a needle retaining guide disposed in proximity to the barrel retaining guide and including an elongated tapered aperture configured to selectively receive a needle of the syringe; and a lid hingedly coupled to the main body and including a substantially planar top cover including a right side flange and a left side flange that extend orthogonally from an opposing right edge and a left edge of the top cover, wherein a latch is formed in each of the right side flange and the left side flange that are configured to engage with a complementary latch disposed on each of a right side wall and a left side wall of the main body to secure the lid to the main body in a closed position.

In some embodiments, a syringe and needle guard assembly is disclosed as comprising: a syringe including a barrel, a plunger disposed within the barrel, and a needle coupled to and extending from a nose of the barrel; and a needle guard, including: a main body forming an open top receiving enclosure configured to at least partially receive the syringe, wherein the main body includes an open front portion including an opening formed in a front wall, a barrel retaining guide disposed in proximity to the front wall and including an arcuate aperture configured to engage a portion of the barrel of the syringe, and a needle retaining guide disposed in proximity to the barrel retaining guide and including an elongated tapered aperture configured to selectively receive the needle of the syringe; and a lid hingedly coupled to the main body and including a substantially planar top cover including a right side flange and a left side flange that extend orthogonally from an opposing right edge and a left edge of the top cover, wherein a latch is formed in each of the right side flange and the left side flange that are configured to engage with a complementary latch disposed on each of a right side wall and a left side wall of the main body to secure the lid to the main body in a closed position and retain the syringe in the needle guard.

### BRIEF DESCRIPTION OF THE DRAWINGS

For a more complete understanding of the present disclosure and so that the manner in which the features and advantages of the embodiments can be understood in more detail, reference is now made to the following brief description, taken in connection with the accompanying drawings and detailed description.

FIG. 1 shows an oblique view of a syringe according to an embodiment of the disclosure.

FIG. 2A shows an oblique view of a needle guard in an open position according to an embodiment of the disclosure.

FIG. 2B shows an orthogonal top view of the needle guard in the open position according to an embodiment of the disclosure.

FIG. 2C shows an orthogonal right-side view of the needle guard in the open position according to an embodiment of the disclosure.

FIG. 2D shows an orthogonal front view of the needle guard in the open position according to an embodiment of the disclosure.

FIG. 3A shows an oblique view of a syringe assembly in an open position according to an embodiment of the disclosure.

FIG. 3B shows an orthogonal top view of the syringe assembly with the needle guard in the open position according to an embodiment of the disclosure.

FIG. 3C shows an orthogonal right-side view of the syringe assembly with the needle guard in the open position according to an embodiment of the disclosure.

FIG. 3D shows an orthogonal right-side view of a syringe assembly with the needle guard in a closed position according to an embodiment of the disclosure.

FIG. 4 shows a simplified schematic diagram of a bulk needle guard holder according to an embodiment of the disclosure.

The use of the same reference symbols in different drawings indicates similar or identical items.

### DETAILED DESCRIPTION

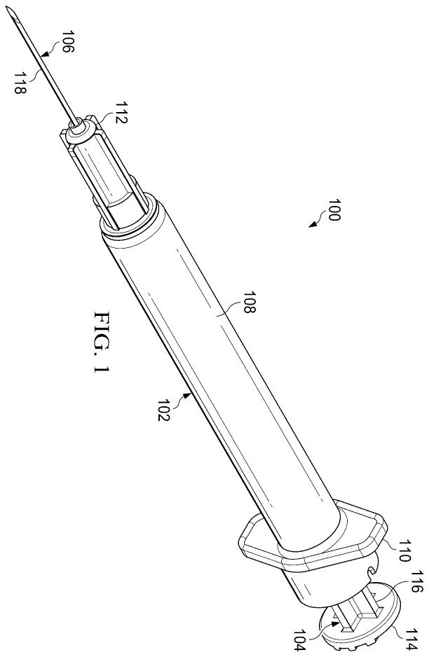

Referring to FIG. 1, an oblique view of a syringe 100 is shown according to an embodiment of the disclosure. The syringe 100 may generally comprise a barrel 102, a plunger assembly 104 disposed within and slidably engaging the barrel 102, and a needle assembly 106. The barrel 102 may comprise a cylindrical body portion 108 that defines a fluid chamber within the barrel 102, one or more annularly or laterally projecting finger flanges 110 disposed at a rearward end of the barrel 102, and a nose 112 disposed at a forward end of the barrel 102 for receiving the needle assembly 106. The plunger assembly 104 may comprise a plunger surface 114 for selectively actuating the plunger assembly 104, an elongated plunger body 116 extending therefrom and at least partially received within the fluid chamber, and a plunger seal (not shown) disposed on or about a distal end of the elongated plunger body 116. The plunger seal may be configured to form and maintain a fluid tight seal between the plunger assembly 104 and the fluid chamber of the barrel 102 during use of the syringe 100. The needle assembly 106 may comprise a needle 118 received within and extending forwardly from the nose 112 of the barrel 102. In some embodiments, the needle assembly 106 may also comprise needle holder or hub (not shown) that retains and/or secures the needle 118 within the nose 112 of the barrel and in fixed relation to the barrel 102 when syringe 100 is fully assembled and/or used.

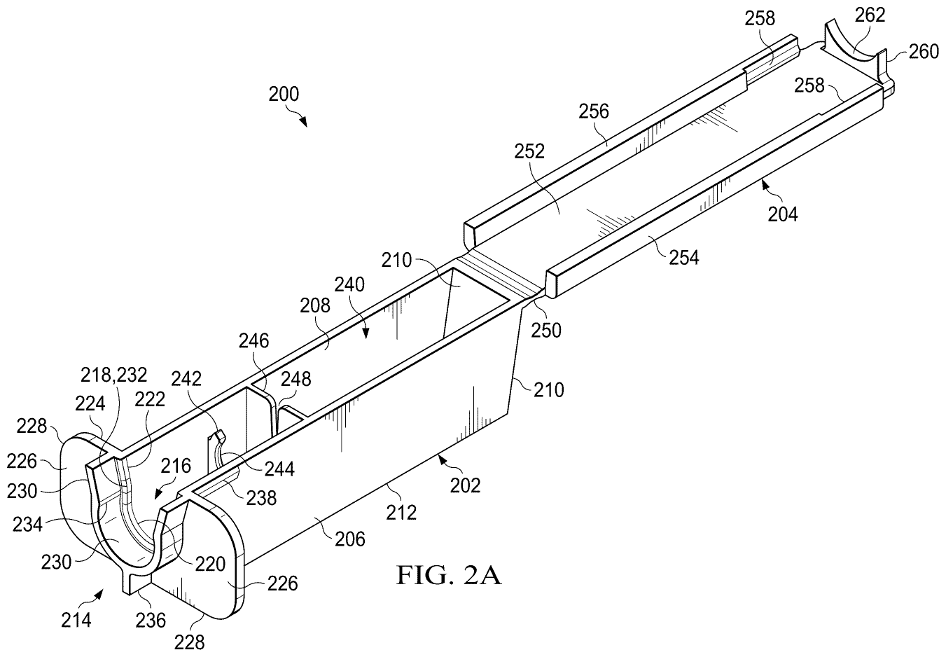

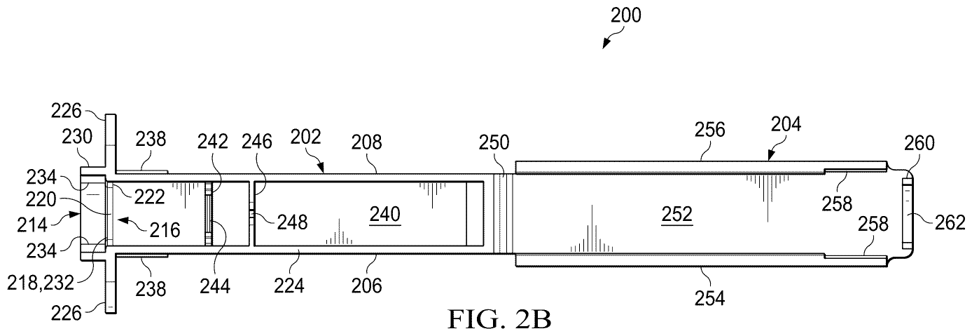

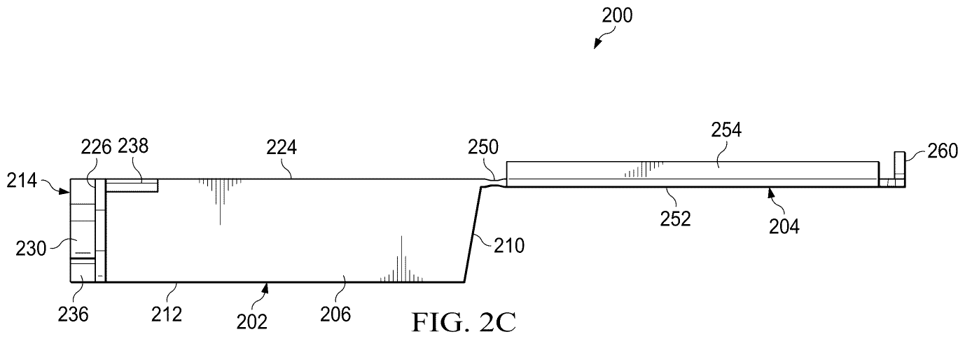

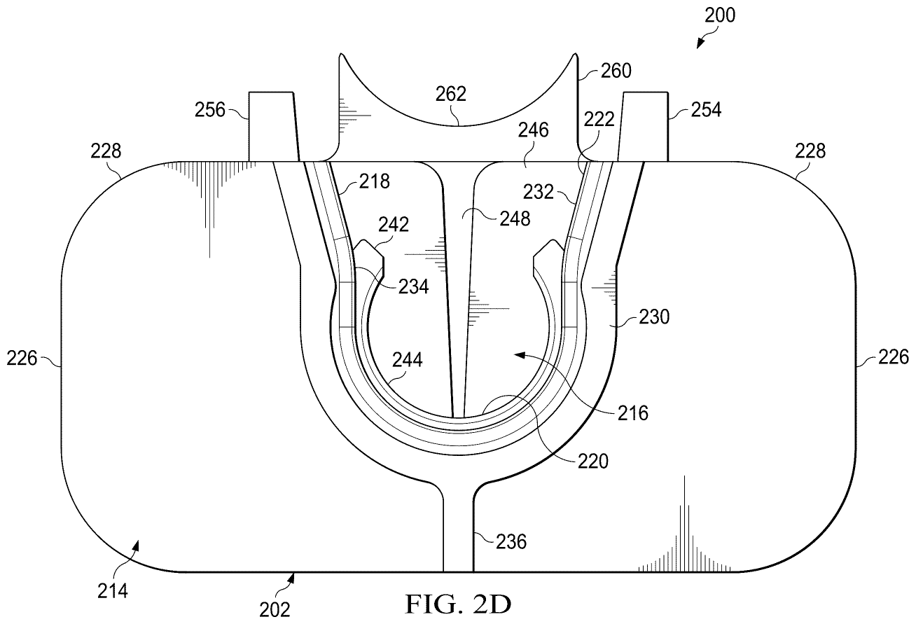

Referring now to FIGS. 2A to 2D, an oblique view, an orthogonal top view, a right-side view, and a front view of a needle guard 200 are shown in an open position according to an embodiment of the disclosure. The needle guard 200 may generally comprise a main body 202 and a lid 204 flexibly or hingedly coupled thereto to allow the lid to articulate between the open position and a closed position. The main body 202 and the lid 204 may generally be unitarily molded, such that the needle guard 200 may be formed by a manufacturing process such as injection molding. In some embodiments, the needle guard 200 may be formed from a thermoplastic or thermoset polymer. The main body 202 may comprise a receiving enclosure configured to at least partially receive a syringe 100 therein. The main body 202 may comprise a right side wall 206, a left side wall 208, a rear wall 210, and a bottom wall 212. In some embodiments, the right side wall 206 and the left side wall 208 may be substantially parallel and oriented perpendicularly to the bottom wall 212. In some embodiments, the rear wall 210 may be oriented perpendicularly to the bottom wall 212. In some embodiments, the rear wall 210 may be oriented greater than perpendicularly to the bottom wall 212, such that the rear wall 210 forms an obtuse angle with the bottom wall 212.

The main body 202 may also comprise an open front portion 214. The front portion 214 may comprise an opening 216 for receiving a syringe 100 therein. The opening 216 may be formed in a front wall 218 of the main body 202. The opening 216 may comprise an arcuate portion 220 and a flared entry portion 222. The arcuate portion 220 may comprise a semicircular profile that extends arcuately about one hundred eighty degrees. The flared entry portion 222 may extend outwardly from the arcuate portion 220 to form a wider entry portion of the opening 216 through which a syringe 100 may be received. The flared entry portion 222 may extend from the arcuate portion 220 to a top edge 224 of the main body 202 to form a passageway that allows a syringe 100 to be selectively received from a top of the main body 202 through the flared entry portion 222 and into the arcuate portion 220 of the opening 216. In some embodiments, the flared entry portion 222 may comprise opposing linear sides. In some embodiments, the flared entry portion 222 may comprise opposing arcuate sides that curve outwardly in an opposing direction as compared to the arcuate portion 220.

The front portion 214 may also comprise a flange 226 that extends laterally outward from each of the right side wall 206 and the left side wall 208. The flange 226 may extend along an entire vertical length of each of the right side wall 206 and the left side wall 208, such that the flange traverses the entire height of the main body 202. In some embodiments, the flange 226 may comprise rounded or beveled corners 228. The flange 226 may generally be configured to aid in removing a syringe 100 from the needle guard 200 when the lid 204 is secured to the main body 202 and the syringe 100 is disposed within the needle guard 200. The front portion 214 may also comprise a collar 230 that projects forwardly from the flange 226 and borders the opening 216. In some embodiments, the collar 230 may comprise a complementary profile to the opening 216. In some embodiments, the collar 230 may be sized so that an inner lip 232 formed by an exposed section of the front wall 218 protrudes inwardly from the collar 230. In some embodiments, the collar 230 may comprise inwardly facing retaining features 234, such as prongs, tines, or ribs that extend inwardly from opposing sides of the collar 230 and are configured to engage a barrel 102 of a syringe 100 to retain the syringe 100 within the needle guard 200. Further, in some embodiments, the front portion 214 may also comprise a reinforcing rib 236 that extends radially from a bottom side of the collar to the bottom wall 212.

The main body 202 may also comprise a latch 238 that extends rearwardly from the flange 226 along each of the right side wall 206 and the left side wall 208 and adjacently along the top edge 224. The latches 238 may comprise angled or inclined ridges that are configured to engage with complementary features on the lid 204 to latch or secure the lid 204 to the main body 202 in the closed position. In some embodiments, the latches 238 may extend only partially along a length of the right side wall 206 and the left side wall 208. In some embodiments, the latches 238 may extend along an entire length of the right side wall 206 and the left side wall 208 to the rear wall 210.

Collectively, the walls 206, 208, 210, the bottom wall 212, and the front portion 214 may be integrally formed to form the receiving enclosure of the main body 202, which comprises an internal compartment or cavity 240. The main body 202 and/or cavity 240 may comprise a barrel retaining guide 242 disposed in proximity to the front wall 218. The barrel retaining guide 242 may extend between the right side wall 206 and the left side wall 208 and to the bottom wall 212. The barrel retaining guide 242 may also not extend to the top edge 224. The barrel retaining guide 242 may comprise an arcuate aperture 244 that extends arcuately greater than one hundred eighty degrees. The arcuate aperture 244 may be configured to engage a portion (e.g., barrel 102, nose 112) of the syringe 100 disposed within the cavity 240 and extend greater than one hundred eighty degrees around the portion of the syringe 100 to securely retain the syringe 100 within the cavity 240 of the main body 202. In some embodiments, the barrel retaining guide 242 may cooperate with the opening 216 to securely retain and prevent lateral and/or vertical movement of the syringe 100 within the needle guard 200.

The main body 202 and/or cavity 240 may comprise a needle retaining guide 246 disposed in proximity to the barrel retaining guide 242 and between the barrel retaining guide 242 and the rear wall 210. The needle retaining guide 246 may extend between the right side wall 206 and the left side wall 208 and to the bottom wall 212. The needle retaining guide 246 may also extend to the top edge 224. The needle retaining guide 246 may comprise an elongated tapered aperture 248. The elongated tapered aperture 248 may be configured to selectively receive a needle 118 of a syringe 100 when a syringe 100 is disposed within the needle guard 200. In some embodiments, the elongated tapered aperture 248 may be sized to accommodate various sized needles 118 and to prevent the needle tip disposed at the distal end of the needle 118 from contacting the right side wall 206 and the left side wall 208 to prevent damage to the needle 118 during transport or preparation for use. In some embodiments, the needle retaining guide 246 may cooperate with the barrel retaining guide 242 to securely retain and prevent lateral movement or torsion of the syringe 100 within the needle guard 200.

The lid 204 may generally be hingedly coupled to the main body 202 by a flexible hinge 250. In some embodiments, the hinge 250 may couple the lid 204 to the rear wall 210 of the main body 202 along the top edge 224 of the main body 202. The hinge 250 may be integrally formed with the main body 202 and the lid 204 and configured to allow the lid 204 to articulate with respect to the main body 202. In some embodiments, the hinge 250 may comprise a thickness that is thinner than a wall thickness of the main body 202 and/or the lid 204 along which the lid 204 may articulate with respect to the main body 202 to allow the lid 204 to selectively open and close.

The lid 204 may generally comprise a top cover 252 comprising a substantially planar, rectangular profile. The lid 204 may also comprise a right side flange 254 and left side flange 256 that extend from opposing right and left edges of the top cover 252. In some embodiments, the right side flange 254 and left side flange 256 may extend substantially orthogonally from the right and left edges of the top cover 252. In some embodiments, the width of the top cover 252 and/or distance between the right side flange 254 and left side flange 256 may be sized such that the main body 202 is received between the right side flange 254 and left side flange 256 when the lid 204 is articulated to the closed position. A latch 258 may be formed in a distal end of each of the right side flange 254 and left side flange 256. The latches 258 may comprise angled or inclined ridges that are complementary to and configured to engage with the latches 238 on the main body 202 to latch or secure the lid 204 to the main body 202 in the closed position. In some embodiments, the latches 258 may extend only partially along a length of the right side flange 254 and left side flange 256. In some embodiments, the latches 238 may extend along an entire length of the right side flange 254 and left side flange 256 depending on the size and configuration of the needle guard 200.

The lid 204 may also comprise a barrel clamp 260 disposed at a distal end of the top cover 252. In some embodiments, the barrel clamp 260 may extend substantially orthogonally from the distal end of the top cover 252. The barrel clamp 260 may comprise an arcuate recess 262 that is configured to engage a top side of a barrel 102 of a syringe 100 when the syringe 100 is disposed within the needle guard 200 and the lid 204 is in the closed position. In some embodiments, the flared entry portion 222 of the opening 216 may be configured to receive the barrel clamp 260, such that the arcuate recess 262 of the barrel clamp 260 cooperates and/or aligns with the arcuate portion 220 of the opening 216 to cooperatively engage the barrel 102 of the syringe 100. In some embodiments, the collar 230 may be configured to receive the barrel clamp 260, such that the arcuate recess 262 of the barrel clamp 260 is disposed adjacently to and forwardly of the inner lip 232 formed by the exposed section of the front wall 218 of the main body 202 to cooperatively engage the barrel 102 of the syringe 100.

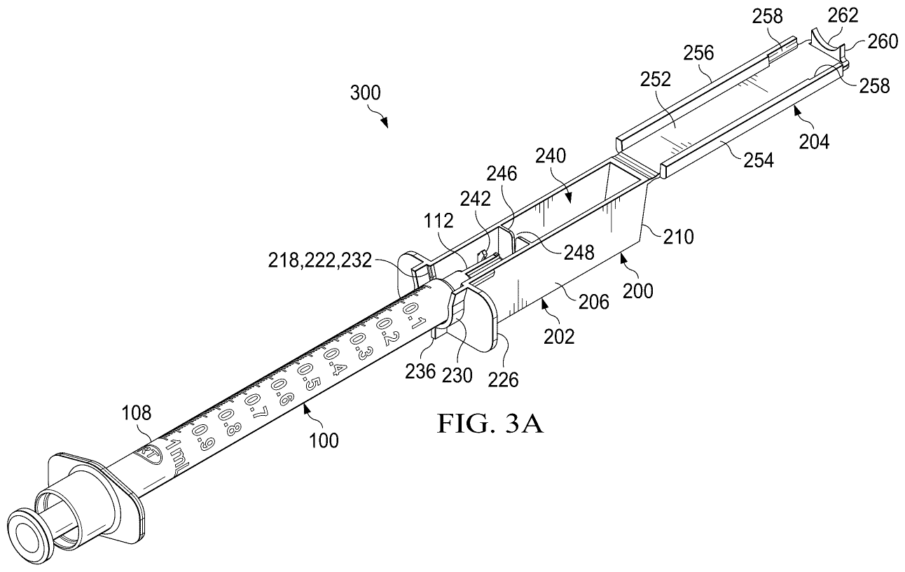

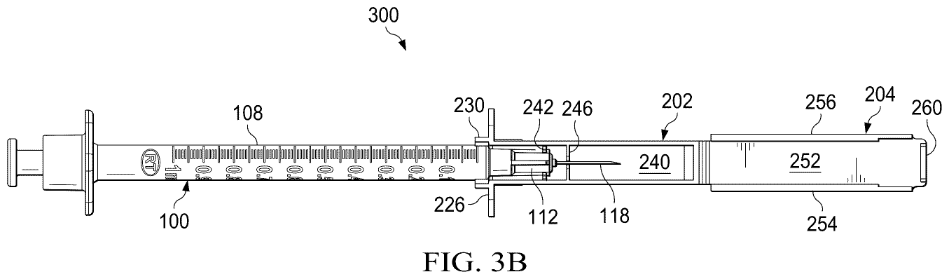

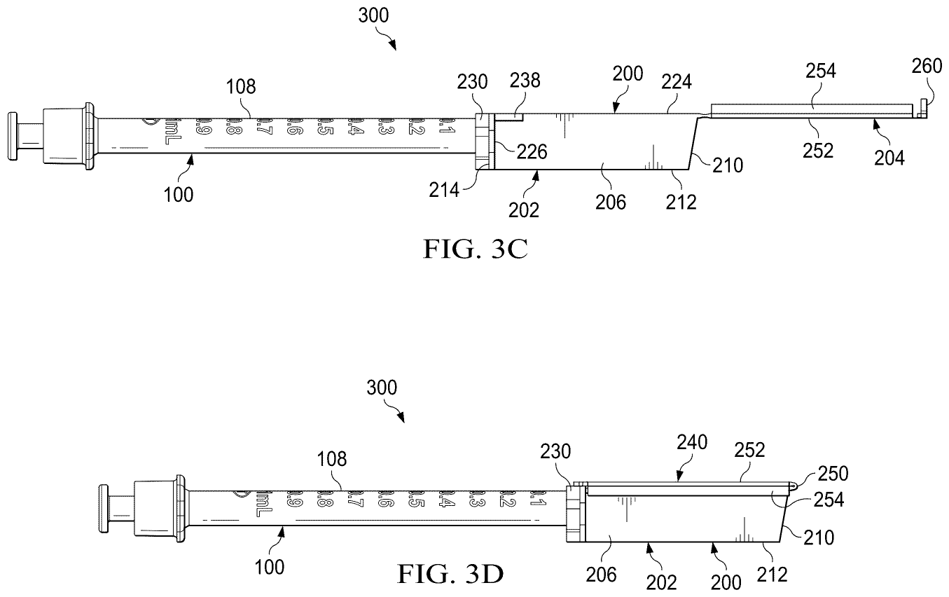

Referring now to FIGS. 3A to 3D, an oblique view, an orthogonal top view, a right-side open view, and a right-side closed view of a syringe assembly 300 are shown according to an embodiment of the disclosure. The syringe assembly 300 may generally comprise a syringe 100 and a needle guard 200 coupled to the syringe 100. The needle guard 200 may be configured to at least partially receive a syringe 100 therein to protect the needle 118 of the syringe 100 from damage during transport to and/or storage at a medical facility prior to use. With the needle guard 200 in the open position (as shown in FIGS. 3A to 3C) the syringe 100 may be placed into the needle guard 200 from the top. The needle 118 of the syringe 100 may be received within the elongated tapered aperture 248 formed in the needle retaining guide 246. The elongated tapered aperture 248 may be sized to accommodate various sized needles 118 and to prevent the needle tip disposed at the distal end of the needle 118 from contacting the right side wall 206 and the left side wall 208 to prevent damage to the needle 118 during transport or preparation for use.

The barrel 102 of the syringe 100 may also be received within and/or engaged with the arcuate aperture 244 of the barrel retaining guide 242. In some embodiments, the barrel retaining guide 242 may be configured and/or positioned such that the arcuate aperture 244 engages the nose 112 of the barrel 102 of the syringe 100. In other embodiments, the barrel retaining guide 242 may be configured and/or positioned such that the arcuate aperture 244 engages another portion of the barrel 102 of the syringe 100. The arcuate aperture 244 may extend greater than one hundred eighty degrees around the engaged portion of the syringe 100 to securely retain the syringe 100 within the cavity 240 of the main body 202. In some embodiments, an opening of the arcuate aperture 244 may comprise an interference fit such that the retained portion of the syringe 100 snaps or locks into the arcuate aperture 244 to restrict and/or altogether prevent lateral and/or vertical movement of the syringe 100 within the needle guard 200.

The barrel 102 of the syringe 100 may also be received within the opening 216 of the front portion 214 of the main body 202 and extend externally therefrom. The flared entry portion 222 may promote easy insertion and/or alignment of the syringe 100 with the opening 216 and may form a passageway that allows a syringe 100 to be selectively received from a top of the main body 202 through the flared entry portion 222 and into the arcuate portion 220 of the opening 216. In some embodiments, the inwardly facing retaining features 234 disposed on opposing sides of the collar 230 may comprise prongs, tines, or ribs that engage the barrel 102 of the syringe 100 to retain the syringe 100 within the needle guard 200. In some embodiments, the collar 230 that projects forwardly from the flange 226 and borders the opening 216 may also support the syringe 100 while installed in the needle guard 200. Collectively, the elongated tapered aperture 248 formed in the needle retaining guide 246, the arcuate aperture 244 formed in the barrel retaining guide 242, and the opening 216 and/or the inwardly facing retaining features 234 disposed on opposing sides of the collar 230 may cooperate to securely retain the syringe 100 within the needle guard 200 and prevent lateral and/or vertical movement of the syringe 100 within the needle guard 200.

Once the syringe 100 is disposed within the needle guard 200, the lid may be closed to secure the syringe 100 in the needle guard 200. When the lid 204 is traversed about the hinge 250 to the closed position (as shown in FIG. 3D), the main body 202 may be received between the right side flange 254 and left side flange 256, and the top edge 224 of the main body may contact the top cover 252 of the lid 204. The latches 258 of the lid 204 may also come into contact with the complementary latches 238 on the main body 202 and engage to secure the lid 204 to the main body 202 in the closed position. Further, the barrel clamp 260 disposed at a distal end of the top cover 252 may be received within the flared entry portion 222 of the opening 216 and/or the collar 230, such that the arcuate recess 262 formed in the barrel clamp 260 cooperatively engages a top side of the barrel 102 of the syringe 100 to securely retain the syringe 100 within the needle guard 200 and prevent lateral and/or vertical movement of the syringe 100 within the needle guard 200. In this manner, the needle guard 200 is tamper resistant and cannot be reopened without contorting or manipulating the needle guard 200 outside the scope of normal use and/or destroying the structural integrity of the needle guard 200. However, in alternative embodiments, the needle guard 200 may not comprise a lid 204 and comprise an open top main body 202 that forms the needle guard 200.

In some embodiments, one or more of the retaining features (e.g., elongated tapered aperture 248 arcuate aperture 244, inwardly facing retaining features 234, and/or opening 216) may frictionally engage the syringe 100 to prevent the syringe 100 from unexpectedly or undesirably falling out of the needle guard 200. This effectively retains the syringe 100 within the needle guard 200 and prevents the syringe 100 from being removed from the needle guard 200 through the opening 216 without a requisite extraction force applied to the syringe 100. As such, the needle guard 200 may be configured to allow a user to remove the syringe 100 from the needle guard 200 by applying a longitudinal force to the syringe 100 to overcome the frictional force applied to the syringe 100 by the one of more retaining features and remove the syringe 100 through the opening 216 by longitudinally sliding the syringe 100 through the opening 216. Additionally, in some embodiments, the flange 226 may aid in removing the syringe 100 from the needle guard 200 by applying an opposing longitudinal force to the flange 226.

The needle guard 200 may generally be formed from a thermoplastic or thermoset elastomer. In some embodiments, the needle guard 200 may be transparent to allow inspection of the syringe 100 through the lid 204 or other portion of the needle guard 200. In some embodiments, one or more components (e.g., main body 202, lid 204) may be color coded to identify one or more characteristics (e.g., size, needle size, application) of the syringe 100. For example, a transparent orange needle guard 200 may represent that the needle guard 200 contains an insulin syringe 100. Further, in some embodiments, the needle guard 200 may be configured to readily accept a sticker that identifies one or more characteristics of the syringe. In some embodiments, the needle guard 200 may be sized and configured to work with a specific type and/or size of syringe 100. In some embodiments, the needle guard 200 may be sized and configured to work with various types and/or sizes of syringes 100. In some embodiments, the needle guard 200 may be sized and configured to comprise a universal fit with all syringes 100.

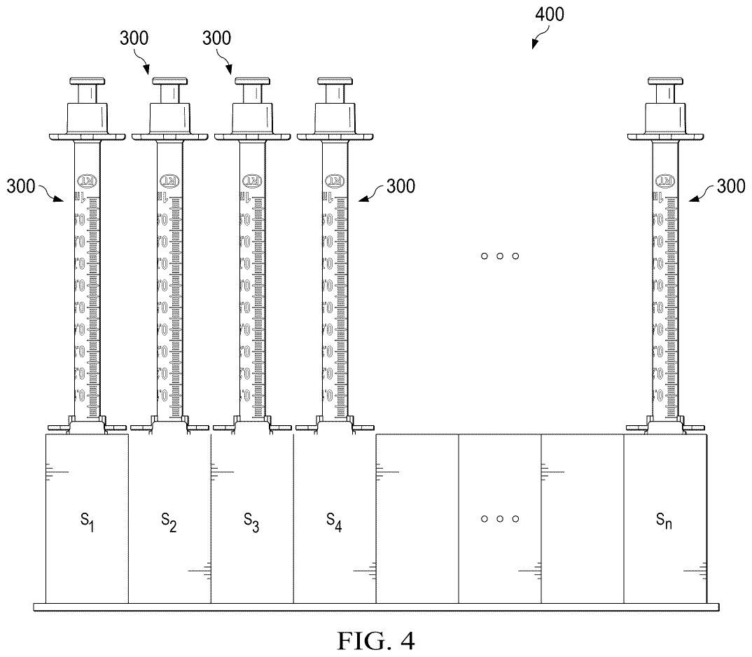

Referring to FIG. 4, a simplified schematic diagram of a bulk needle guard holder 400 is shown according to an embodiment of the disclosure. The bulk needle guard holder 400 may generally be configured to systematically carry a plurality of syringe assemblies 300, each syringe assembly comprising a syringe 100 and a needle guard 200, during transport to or storage at a medical facility prior to use. The bulk needle guard holder 400 provides safe, efficient, and compact storage organization for the plurality of syringe assemblies 300 to further prevent damage to the needles 118 of the syringes 100. As shown, each of S₁, S₂, S₃, and Sₙ represent a syringe assembly 300, where “n” represents any number depending on the capacity of the bulk needle guard holder 400. Further, while each syringe assembly 300 is shown as comprising the same size of syringe 100 and needle guard 200, it will be appreciated that the bulk needle guard holder 400 may comprise the versatility of organizing and storing a plurality of syringe assemblies 300 having different sized syringes 100 and/or needle guards 200.

It will be appreciated that a syringe 100, a needle guard 200, and/or a syringe assembly 300 disclosed herein may include one or more of the following embodiments:
Embodiment 1. A needle guard, comprising: A needle guard, comprising: a main body forming an open top receiving enclosure configured to at least partially receive a syringe, wherein the main body comprises an open front portion comprising an opening formed in a front wall, a barrel retaining guide disposed in proximity to the front wall and comprising an arcuate aperture configured to engage a portion of a barrel of the syringe, and a needle retaining guide disposed in proximity to the barrel retaining guide and comprising an elongated tapered aperture configured to selectively receive a needle of the syringe; and a lid hingedly coupled to the main body and comprising a substantially planar top cover comprising a right side flange and a left side flange that extend orthogonally from an opposing right edge and a left edge of the top cover, wherein a latch is formed in each of the right side flange and the left side flange that are configured to engage with a complementary latch disposed on each of a right side wall and a left side wall of the main body to secure the lid to the main body in a closed position.
Embodiment 2. The needle guard of embodiment 1, wherein the opening comprises an arcuate portion and a flared entry portion.
Embodiment 3. The needle guard of embodiment 2, wherein the arcuate portion comprises a semicircular profile that extends arcuately about one hundred eighty degrees.
Embodiment 4. The needle guard of embodiment 3, wherein the flared entry portion extends outwardly from the arcuate portion to a top edge of the main body to form a passageway that allows the syringe to be selectively received from a top of the main body through the flared entry portion and into the arcuate portion of the opening.
Embodiment 5. The needle guard of embodiment 1, wherein the front portion comprises a collar that borders the opening.
Embodiment 6. The needle guard of embodiment 5, wherein the collar comprises opposing retaining features that extend inwardly from opposing sides of the collar and are configured to engage the barrel of the syringe to retain the syringe within the needle guard.
Embodiment 7. The needle guard of embodiment 1, wherein the barrel retaining guide extends between the right side wall and the left side wall.
Embodiment 8. The needle guard of embodiment 7, wherein the arcuate aperture of the barrel retaining guide extends arcuately greater than one hundred eighty degrees.
Embodiment 9. The needle guard of embodiment 8, wherein the barrel retaining guide is positioned within the main body such that the arcuate aperture engages a nose of the barrel of the syringe.
Embodiment 10. The needle guard of embodiment 1, wherein the needle retaining guide is disposed between the barrel retaining guide and a rear wall of the main body.
Embodiment 11. The needle guard of embodiment 1, wherein the needle retaining guide extends between the right side wall and the left side wall.
Embodiment 12. The needle guard of embodiment 11, wherein the needle retaining guide cooperates with the barrel retaining guide to securely retain and prevent lateral movement of the syringe within the needle guard.
Embodiment 13. The needle guard of embodiment 1, wherein the right side wall and the a left side wall are substantially parallel and oriented perpendicularly to a bottom wall of the main body.
Embodiment 14. The needle guard of embodiment 13, wherein the main body comprises a rear wall oriented greater than perpendicularly to the bottom wall.
Embodiment 15. The needle guard of embodiment 14, wherein the lid is hingedly coupled to the main body by a flexible hinge, and wherein the hinge couples the top cover of the lid to the rear wall of the main body along a top edge of the main body.
Embodiment 16. The needle guard of embodiment 15, wherein the hinge comprises a thickness that is thinner than a wall thickness of the main body, the lid, or a combination thereof.
Embodiment 17. The needle guard of embodiment 1, wherein the complementary latches are disposed on each of the right side wall and the left side wall of the main body extend rearwardly along each of the right side wall and the left side wall and adjacently along the top edge.
Embodiment 18. The needle guard of embodiment 17, wherein the complementary latches extend rearwardly from a flange that extends laterally from each of the right side wall and the left side wall of the main body from the front wall of the main body.
Embodiment 19. The needle guard of embodiment 18, wherein the complementary latches comprise angled or inclined ridges that are configured to engage with the latches on the lid to secure the lid to the main body in the closed position.
Embodiment 20. The needle guard of embodiment 1, wherein the open top receiving enclosure is configured to receive the syringe from a top of the main body.
Embodiment 21. The needle guard of embodiment 1, wherein the main body is received between the right side flange and the left side flange of the lid when the lid is in the closed position.
Embodiment 22. The needle guard of embodiment 21, wherein the lid comprises a barrel clamp disposed at a distal end of the top cover, and wherein the barrel clamp comprises an arcuate recess configured to engage a top side of the barrel of the syringe when the lid is in the closed position.
Embodiment 23. The needle guard of embodiment 6, wherein the opening, the elongated tapered aperture, the arcuate aperture, the opposing retaining features, or any combination thereof are configured to frictionally engage the syringe to prevent the syringe from falling out of the needle guard without a requisite longitudinal extraction force applied to the syringe when the needle guard is in the closed position.
Embodiment 24. The needle guard of embodiment 23, wherein the needle guard is configured to allow a user to remove the syringe from the needle guard through the opening by applying the requisite longitudinal extraction force to the syringe to overcome the frictional force applied by the needle guard to the syringe.
Embodiment 25. The needle guard of embodiment 1, wherein the main body and the lid are unitarily molded.
Embodiment 26. The needle guard of embodiment 25, wherein the needle guard is formed from a thermoplastic or thermoset elastomer.
Embodiment 27. The needle guard of embodiment 26, wherein the needle guard is transparent to allow inspection of the syringe through the lid or other portion of the needle guard.
Embodiment 28. The needle guard of embodiment 27, wherein the main body, the lid, or a combination thereof are color coded to identify one or more characteristics of the syringe contained in the needle guard.
Embodiment 29. A syringe and needle guard assembly, comprising: a syringe comprising a barrel, a plunger disposed within the barrel, and a needle coupled to and extending from a nose of the barrel; and a needle guard, comprising: a main body forming an open top receiving enclosure configured to at least partially receive the syringe, wherein the main body comprises an open front portion comprising an opening formed in a front wall, a barrel retaining guide disposed in proximity to the front wall and comprising an arcuate aperture configured to engage a portion of the barrel of the syringe, and a needle retaining guide disposed in proximity to the barrel retaining guide and comprising an elongated tapered aperture configured to selectively receive the needle of the syringe; and a lid hingedly coupled to the main body and comprising a substantially planar top cover comprising a right side flange and a left side flange that extend orthogonally from an opposing right edge and a left edge of the top cover, wherein a latch is formed in each of the right side flange and the left side flange that are configured to engage with a complementary latch disposed on each of a right side wall and a left side wall of the main body to secure the lid to the main body in a closed position and retain the syringe in the needle guard.
Embodiment 30. The syringe and needle guard assembly of embodiment 29, wherein the opening comprises an arcuate portion and a flared entry portion.
Embodiment 31. The syringe and needle guard assembly of embodiment 30, wherein the arcuate portion comprises a semicircular profile that extends arcuately about one hundred eighty degrees.
Embodiment 32. The syringe and needle guard assembly of embodiment 31, wherein the flared entry portion extends outwardly from the arcuate portion to a top edge of the main body to form a passageway that allows the syringe to be selectively received from a top of the main body through the flared entry portion and into the arcuate portion of the opening.
Embodiment 33. The syringe and needle guard assembly of embodiment 29, wherein the front portion comprises a collar that borders the opening.
Embodiment 34. The syringe and needle guard assembly of embodiment 33, wherein the collar comprises opposing retaining features that extend inwardly from opposing sides of the collar and are configured to engage the barrel of the syringe to retain the syringe within the needle guard.
Embodiment 35. The syringe and needle guard assembly of embodiment 29, wherein the barrel retaining guide extends between the right side wall and the left side wall.
Embodiment 36. The syringe and needle guard assembly of embodiment 35, wherein the arcuate aperture of the barrel retaining guide extends arcuately greater than one hundred eighty degrees.
Embodiment 37. The syringe and needle guard assembly of embodiment 36, wherein the barrel retaining guide is positioned within the main body such that the arcuate aperture engages a nose of the barrel of the syringe.
Embodiment 38. The syringe and needle guard assembly of embodiment 29, wherein the needle retaining guide is disposed between the barrel retaining guide and a rear wall of the main body.
Embodiment 39. The syringe and needle guard assembly of embodiment 29, wherein the needle retaining guide extends between the right side wall and the left side wall.
Embodiment 40. The syringe and needle guard assembly of embodiment 39, wherein the needle retaining guide cooperates with the barrel retaining guide to securely retain and prevent lateral movement of the syringe within the needle guard.
Embodiment 41. The syringe and needle guard assembly of embodiment 29, wherein the right side wall and the a left side wall are substantially parallel and oriented perpendicularly to a bottom wall of the main body.
Embodiment 42. The syringe and needle guard assembly of embodiment 41, wherein the main body comprises a rear wall oriented greater than perpendicularly to the bottom wall.
Embodiment 43. The syringe and needle guard assembly of embodiment 42, wherein the lid is hingedly coupled to the main body by a flexible hinge, and wherein the hinge couples the top cover of the lid to the rear wall of the main body along a top edge of the main body.
Embodiment 44. The syringe and needle guard assembly of embodiment 43, wherein the hinge comprises a thickness that is thinner than a wall thickness of the main body, the lid, or a combination thereof.
Embodiment 45. The syringe and needle guard assembly of embodiment 29, wherein the complementary latches are disposed on each of the right side wall and the left side wall of the main body extend rearwardly along each of the right side wall and the left side wall and adjacently along the top edge.
Embodiment 46. The syringe and needle guard assembly of embodiment 45, wherein the complementary latches extend rearwardly from a flange that extends laterally from each of the right side wall and the left side wall of the main body from the front wall of the main body.
Embodiment 47. The syringe and needle guard assembly of embodiment 46, wherein the complementary latches comprise angled or inclined ridges that are configured to engage with the latches on the lid to secure the lid to the main body in the closed position.
Embodiment 48. The syringe and needle guard assembly of embodiment 29, wherein the open top receiving enclosure is configured to receive the syringe from a top of the main body.
Embodiment 49. The syringe and needle guard assembly of embodiment 29, wherein the main body is received between the right side flange and the left side flange of the lid when the lid is in the closed position.
Embodiment 50. The syringe and needle guard assembly of embodiment 49, wherein the lid comprises a barrel clamp disposed at a distal end of the top cover, and wherein the barrel clamp comprises an arcuate recess configured to engage a top side of the barrel of the syringe when the lid is in the closed position.
Embodiment 51. The syringe and needle guard assembly of embodiment 34, wherein the opening, the elongated tapered aperture, the arcuate aperture, the opposing retaining features, or any combination thereof are configured to frictionally engage the syringe to prevent the syringe from falling out of the needle guard without a requisite longitudinal extraction force applied to the syringe when the needle guard is in the closed position.
Embodiment 52. The syringe and needle guard assembly of embodiment 51, wherein the needle guard is configured to allow a user to remove the syringe from the needle guard through the opening by applying the requisite longitudinal extraction force to the syringe to overcome the frictional force applied by the needle guard to the syringe.
Embodiment 53. The syringe and needle guard assembly of embodiment 28, wherein the main body and the lid are unitarily molded.
Embodiment 54. The syringe and needle guard assembly of embodiment 53, wherein the needle guard is formed from a thermoplastic or thermoset elastomer.
Embodiment 55. The syringe and needle guard assembly of embodiment 54, wherein the needle guard is transparent to allow inspection of the syringe through the lid or other portion of the needle guard.
Embodiment 56. The syringe and needle guard assembly of embodiment 55, wherein the main body, the lid, or a combination thereof are color coded to identify one or more characteristics of the syringe contained in the needle guard.

In the foregoing specification, the concepts have been described with reference to specific embodiments. However, those of ordinary skill in the art appreciate that various modifications and changes can be made without departing from the scope of the invention as set forth in the claims below. Other examples that occur to those skilled in the art are intended to be within the scope of the claims if they have structural elements that do not differ from the literal language of the claims, or if they include equivalent structural elements with insubstantial differences from the literal languages of the claims. Accordingly, the specification and figures are to be regarded in an illustrative rather than in a restrictive sense, and all such modifications are intended to be included within the scope of the invention.

Benefits, other advantages, and solutions to problems have been described above with respect to one or more specific embodiments. After reading the specification, those of ordinary skill in the art will appreciate that certain features that are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range and include sub-ranges that overlap from one preferred range to another preferred range.

What is claimed is:

1. A needle guard, comprising:
a main body forming an open top receiving enclosure configured to at least partially receive a syringe, wherein the main body comprises an open front portion comprising an opening formed in a front wall, a barrel retaining guide disposed in proximity to the front wall and comprising an arcuate aperture configured to engage a portion of a barrel of the syringe, and a needle retaining guide disposed in proximity to the barrel retaining guide and comprising an elongated tapered aperture configured to selectively receive a needle of the syringe; and
a lid hingedly coupled to the main body and comprising a substantially planar top cover comprising a right side flange and a left side flange that extend orthogonally from an opposing right edge and a left edge of the top cover, wherein a latch is formed in each of the right side flange and the left side flange that are configured to engage with a complementary latch disposed on each of a right side wall and a left side wall of the main body to secure the lid to the main body in a closed position.

2. The needle guard of claim 1, wherein the opening comprises an arcuate portion and a flared entry portion, and wherein the flared entry portion extends outwardly from the arcuate portion to a top edge of the main body to form a passageway that allows the syringe to be selectively received from a top of the main body through the flared entry portion and into the arcuate portion of the opening.

3. The needle guard of claim 1, wherein the front portion comprises a collar that borders the opening, and wherein the collar comprises opposing retaining features that extend inwardly from opposing sides of the collar and are configured to engage the barrel of the syringe to retain the syringe within the needle guard.

4. The needle guard of claim 1, wherein the arcuate aperture of the barrel retaining guide extends arcuately greater than one hundred eighty degrees.

5. The needle guard of claim 4, wherein the barrel retaining guide is positioned within the main body such that the arcuate aperture engages a nose of the barrel of the syringe.

6. The needle guard of claim 5, wherein the needle retaining guide is disposed between the barrel retaining guide and a rear wall of the main body.

7. The needle guard of claim 6, wherein the needle retaining guide cooperates with the barrel retaining guide to securely retain and prevent lateral movement of the syringe within the needle guard.

8. The needle guard of claim 1, wherein the right side wall and the left side wall are substantially parallel and oriented perpendicularly to a bottom wall of the main body.

9. The needle guard of claim 8, wherein the main body comprises a rear wall, wherein the lid is hingedly coupled to the main body by a flexible hinge, and wherein the hinge couples the top cover of the lid to the rear wall of the main body along a top edge of the main body.

10. The needle guard of claim 9, wherein the hinge comprises a thickness that is thinner than a wall thickness of the main body, the lid, or a combination thereof.

11. The needle guard of claim 1, wherein the complementary latches are disposed on each of the right side wall and the left side wall of the main body extend rearwardly along each of the right side wall and the left side wall and adjacently along the top edge.

12. The needle guard of claim 11, wherein the complementary latches extend rearwardly from a flange that extends laterally from each of the right side wall and the left side wall of the main body from the front wall of the main body.

13. The needle guard of claim 12, wherein the complementary latches comprise angled or inclined ridges that are configured to engage with the latches on the lid to secure the lid to the main body in the closed position.

14. The needle guard of claim 1, wherein the open top receiving enclosure is configured to receive the syringe from a top of the main body.

15. The needle guard of claim 1, wherein the main body is received between the right side flange and the left side flange of the lid when the lid is in the closed position.

16. The needle guard of claim 15, wherein the lid comprises a barrel clamp disposed at a distal end of the top cover, and wherein the barrel clamp comprises an arcuate recess configured to engage a top side of the barrel of the syringe when the lid is in the closed position.

17. The needle guard of claim 16, wherein the needle guard is configured to allow a user to remove the syringe from the needle guard through the opening by applying a requisite longitudinal extraction force to the syringe to overcome a frictional force applied by the needle guard to the syringe.

18. The needle guard of claim 1, wherein the main body and the lid are unitarily molded.

19. The needle guard of claim 18, wherein the needle guard is transparent to allow inspection of the syringe through the lid or other portion of the needle guard.

20. The needle guard of claim 18, wherein the main body, the lid, or a combination thereof are color coded to identify one or more characteristics of the syringe contained in the needle guard.

21. A syringe and needle guard assembly, comprising:
a syringe comprising a barrel, a plunger disposed within the barrel, and a needle coupled to and extending from a nose of the barrel; and
a needle guard, comprising:
a main body forming an open top receiving enclosure configured to at least partially receive the syringe, wherein the main body comprises an open front portion comprising an opening formed in a front wall, a barrel retaining guide disposed in proximity to the front wall and comprising an arcuate aperture configured to engage a portion of the barrel of the syringe, and a needle retaining guide disposed in proximity to the barrel retaining guide and comprising an elongated tapered aperture configured to selectively receive the needle of the syringe; and
a lid hingedly coupled to the main body and comprising a substantially planar top cover comprising a right side flange and a left side flange that extend orthogonally from an opposing right edge and a left edge of the top cover, wherein a latch is formed in each of the right side flange and the left side flange that are configured to engage with a complementary latch disposed on each of a right side wall and a left side wall of the main body to secure the lid to the main body in a closed position and retain the syringe in the needle guard.

22. The syringe and needle guard assembly of claim 21, wherein the opening comprises an arcuate portion and a flared entry portion, and wherein the flared entry portion extends outwardly from the arcuate portion to a top edge of the main body to form a passageway that allows the syringe to be selectively received from a top of the main body through the flared entry portion and into the arcuate portion of the opening.

23. The syringe and needle guard assembly of claim 21, wherein the front portion comprises a collar that borders the opening, and wherein the collar comprises opposing retaining features that extend inwardly from opposing sides of the collar and are configured to engage the barrel of the syringe to retain the syringe within the needle guard.

24. The syringe and needle guard assembly of claim 21, wherein the arcuate aperture of the barrel retaining guide extends arcuately greater than one hundred eighty degrees, and wherein the barrel retaining guide is positioned within the main body such that the arcuate aperture engages a nose of the barrel of the syringe.

25. The syringe and needle guard assembly of claim 21, wherein the main body comprises a rear wall, wherein the lid is hingedly coupled to the main body by a flexible hinge, and wherein the hinge couples the top cover of the lid to the rear wall of the main body along a top edge of the main body.

26. The syringe and needle guard assembly of claim 21, wherein the open top receiving enclosure is configured to receive the syringe from a top of the main body.

27. The syringe and needle guard assembly of claim 21, wherein the lid comprises a barrel clamp disposed at a distal end of the top cover, and wherein the barrel clamp comprises an arcuate recess configured to engage a top side of the barrel of the syringe when the lid is in the closed position.

28. The syringe and needle guard assembly of claim 27, wherein the needle guard is configured to allow a user to remove the syringe from the needle guard through the opening by applying a requisite longitudinal extraction force to the syringe to overcome a frictional force applied by the needle guard to the syringe.

29. The syringe and needle guard assembly of claim 21, wherein the needle guard is transparent to allow inspection of the syringe through the lid or other portion of the needle guard.

30. The syringe and needle guard assembly of claim 21, wherein the main body, the lid, or a combination thereof are color coded to identify one or more characteristics of the syringe contained in the needle guard.
